(19) United States
(12) Patent Application Publication
(10) Pub. No.: US 2010/0004733 A1
(43) Pub. Date: Jan. 7, 2010

(54) Implants Including Fractal Structures

(75) Inventors: Liliana Atanasoska, Edina, MN (US); Tom Holman, Princeton, MN (US); James Q. Feng, Maple Grove, MN (US); Robert W. Warner, Woodbury, MN (US); Afsar Ali, Maple Grove, MN (US)

Correspondence Address: FISH & RICHARDSON P.C., PO BOX 1022, MINNEAPOLIS, MN, 55440-1022, US

(73) Assignee: BOSTON SCIENTIFIC SCIMED, INC., Maple Grove, MN (US)

(21) Appl. No.: 12/166,507

(22) Filed: Jul. 2, 2008

(51) Int. Cl. A61F 2/82

(52) U.S. Cl. 623/1.15

(57) ABSTRACT

An endoprosthesis includes a member having a surface that includes a fractal structure.

### TECHNICAL FIELD

This invention relates to implants, and more particularly to stents.

### BACKGROUND

The body includes various passageways such as arteries, other blood vessels, and other body lumens. These passageways sometimes become occluded or weakened. For example, the passageways can be occluded by a tumor, restricted by plaque, or weakened by an aneurysm. When this occurs, the passageway can be reopened or reinforced, or even replaced, with a medical endoprosthesis. An endoprosthesis is typically a tubular member that is placed in a lumen in the body. Examples of endoprostheses include stents, covered stents, and stent-grafts.

Endoprostheses can be delivered inside the body by a catheter that supports the endoprosthesis in a compacted or reduced-size form as the endoprosthesis is transported to a desired site. Upon reaching the site, the endoprosthesis is expanded, for example, so that it can contact the walls of the lumen.

The expansion mechanism can include forcing the endoprosthesis to expand radially. For example, the expansion mechanism can include the catheter carrying a balloon, which carries a balloon-expandable endoprosthesis. The balloon can be inflated to deform and to fix the expanded endoprosthesis at a predetermined position in contact with the lumen wall. The balloon can then be deflated, and the catheter withdrawn.

In another delivery technique, the endoprosthesis is formed of an elastic material that can be reversibly compacted and expanded, e.g., elastically or through a material phase transition. During introduction into the body, the endoprosthesis is restrained in a compacted condition. Upon reaching the desired implantation site, the restraint is removed, for example, by retracting a restraining device such as an outer sheath, enabling the endoprosthesis to self-expand by its own internal elastic restoring force.

### SUMMARY

An endoprosthesis is described that includes a member having a surface that includes a fractal structure.

A fractal structure includes a rough or fragmented geometric shape that can be subdivided in parts, each part being (at least approximately) a reduced-size copy of the rough or fragmented geometric shape. As used herein, the term “fractal structure” means a structure that includes similar structures at magnification factors of 1,000 and 10,000. In some embodiments, the fractal structure can have a surface area greater than 5 times the surface area of a smooth surface having the same dimensions. In some embodiments, the fractal structure can include a cauliflower-like structure. In some embodiments, the fractal structure can include nanopits.

In some embodiments, the member can include a bioerodable material (e.g., a bioerodable metal or a bioerodable polymer). The bioerodable metal can be magnesium, zinc, iron, or an alloy thereof. The bioerodable polymer can be polydioxanone, polycaprolactone, polygluconate, polylactic acid-polyethylene oxide copolymers, modified cellulose, collagen, poly(hydroxybutyrate), polyanhydride, polyphosphoester, poly(amino acids), poly-L-lactide, poly-D-lactide, polyglycolide, poly(alpha-hydroxy acid), or combinations thereof.

In some embodiments, the member can include a non-biodegradable metal (e.g., stainless steels, platinum enhanced stainless steels, cobalt-chromium alloys, nickel titanium alloys, and combinations thereof).

In some embodiments, the surface that includes the fractal structure can be an outermost surface of the endoprosthesis. In other embodiments, the endoprosthesis can further include a second material overlying the fractal structure. For example, the second material can be a tie layer, a biocompatible coating, a drug-eluting layer, a radiopaque metal or alloy, or a combination thereof.

The endoprosthesis may be provided in the form of a stent. For example, the endoprosthesis can be a bioerodable stent including a bioerodable member having a surface having a fractal structure, where the surface includes iron or an alloy thereof.

An implant is also described that includes a bioerodable material having a surface that includes a fractal structure. For example, the implant can be in the form of a stent, a cochlear implant, a bone screw, a neuron aneurysm coil, a septal defect plug, a venous valve support structure, a pacing lead, a spinal implant support structure, a hip replacement joint, or a inter uterine implant.

The details of one or more embodiments are set forth in the accompanying drawings and the description below. Other features, objects, and advantages will be apparent from the description and drawings, and from the claims.

### DESCRIPTION OF DRAWINGS

FIG. 1. illustrates an exemplary stent.

Like reference symbols in the various drawings indicate like elements.

### DETAILED DESCRIPTION

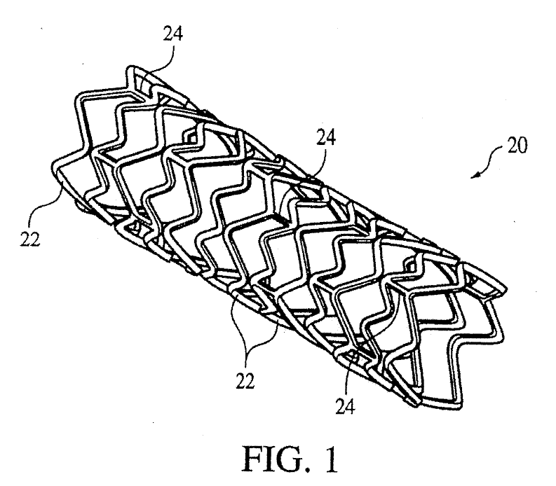

Referring to FIG. 1, a stent 20 can have the form of a tubular member defined by a plurality of bands 22 and a plurality of connectors 24 that extend between and connect adjacent bands. During use, bands 22 can expand from an initial, small diameter compressed state to a larger diameter to contact the stent 20 against a wall of a vessel, thereby maintaining the patency of the vessel. Connectors 24 can provide stent 20 with flexibility and conformability that allow the stent to adapt to the contours of the vessel.

The stent 20 can include a surface that has a fractal structure, as described in the Summary, above. By providing a fractal structure to the surface of a stent, the surface area of the stent can be increased. In some embodiments, the fractal structure can have a surface area greater than 5 times the surface area of a smooth surface having the same dimensions.

The fractal structure on the surface of stent 20 can include a number of fractal structures. For example, the fractal structure can include a cauliflower-like structure at magnification factors of at least 1,000 and 10,000. In some embodiments, the fractal structure can include nanopits. For example, each nanopit can include nanopit walls including smaller nanopits having a similar structure to the larger nanopit, but on a smaller scale. The fractal structure geometry can be tailored to almost any shape/structure that is desired. It can be made to match the stent geometry or it can be made not to match.

Stent 20 can, in some embodiments, include a bioerodable material (e.g., a bioerodable metal or a bioerodable polymer). A bioerodable metal can include magnesium, zinc, iron, or an alloy thereof. In some embodiments, the bioerodable material can be a metallic iron or an alloy thereof (e.g., Fe-35Mn). For example, an iron stent having an outermost surface having a fractal structure could allow for a faster erosion rate. The bioerodable material can also be a bioerodable polymer such as polydioxanone, polycaprolactone, polygluconate, polylactic acid-polyethylene oxide copolymers, modified cellulose, collagen, poly(hydroxybutyrate), polyanhydride, polyphosphoester, poly(amino acids), poly-L-lactide, poly-D-lactide, polyglycolide, poly(alpha-hydroxy acid), or combinations thereof.

Stent 20 can, in some embodiments, include non-biodegradable materials, such as stainless steels, platinum enhanced stainless steels, cobalt-chromium alloys, nickel titanium alloys, or a combination thereof. In some embodiments, stent 20 can include bioerodable and non-bioerodable portions.

Stent 20 can include a uniform distribution of fractal structures. For example, an iron stent body can include a uniform distribution of fractal structures over its entire surface. In other embodiments, stent 20 can include the fractal structure on only an abdominal surface.

Stent 20 can, in some embodiments include regions of preferred erosion. For example, the stent 20 can include select bands 22 or connectors 24, or portions thereof, that include the fractal structure, while the remaining surfaces are smooth. In other embodiments, every surface of the stent body can include the fractal structures, but select bands and/or connectors can include an outer coating to delay the erosion of those select bands and/or connectors.

Stent 20 can, in some embodiments, include a layer of a second material overlying the surface. The layer of second material can overlie at least a portion of the fractal structure. The layer of the second material can be, for example, a tie layer, a biocompatible outer coating, a radiopaque metal or alloy, and/or a drug-eluting layer. Drug eluting layers can be made of biodegradable polymer coatings such as polyesters, polyamide, polyanhidrides, polysaccharides, examples such as PLGA, PLA, Chitosan. Also biological polymers based on rproteins, peptides and amino acids are an option. Besides polymers one can use biodegradable ceramics based on phosphates. For example, a stainless steel stent surface can include a fractal structure and include a layer of a drug-eluting polymer coating over the fractal structure. The presence of the fractal structure could improve the adhesion of a drug-eluting coating to a stainless steel stent surface.

A fractal structure can be formed on a surface of a stent by a number of suitable deposition or patterning treatments, such as plasma enhanced physical vapor deposition, laser etching, and/or chemical etching. By using these processes in a way that reiterates the basic surface structure, a fractal structure can be produced. For example, in plasma-enhanced physical vapor deposition, argon ions from a plasma are accelerated in a high vacuum apparatus by an outside electrical field towards a cathode made of the coating material (e.g., Iron). Single cathode atoms (e.g., iron atoms) can then be sputtered away by the incident argon ions and be deposited on the surface of a stent. By limiting diffusion during the plasma-enhanced physical vapor deposition process, a fractal structure can be obtained.

For example, a fractal surface produced by a diffusion limited plasma-enhanced physical vapor deposition process can have a cauliflower-like appearance at a variety of magnification factors (e.g., at magnification factors between 1,000 and 10,000). An example of a fractal structure having a cauliflower-like structure at both magnification factors of 1,000 and 16,000 can be found in FIGS. 3 and 4 of Schaldach et al., Journal of Material Sciences, Materials in Medicine, 6 (1995) 844.

Chemical etching can also be used to produce a fractal structure, e.g., a fractal structure of nanopits. An example of a chemically etched fractal structure of nanopits is shown in FIG. 5B of Yi et al., Surface Science 600, 2006, 4613.

Stents 10 can be of any desired shape and size (e.g., superficial femoral artery stents, coronary stents, aortic stents, peripheral vascular stents, gastrointestinal stents, urology stents, and neurology stents). Depending on the application, the stent can have a diameter of between, for example, 1 mm to 46 mm. In certain embodiments, a coronary stent can have an expanded diameter of from 2 mm to 6 mm. In some embodiments, a peripheral stent can have an expanded diameter of from 5 mm to 24 mm. In certain embodiments, a gastrointestinal and/or urology stent can have an expanded diameter of from 6 mm to about 30 mm. In some embodiments, a neurology stent can have an expanded diameter of from about 1 mm to about 12 mm. An abdominal aortic aneurysm (AAA) stent and a thoracic aortic aneurysm (TAA) stent can have a diameter from about 20 mm to about 46 mm.

In use, a stent can be used, e.g., delivered and expanded, using a catheter delivery system. Catheter systems are described in, for example, Wang U.S. Pat. No. 5,195,969, Hamlin U.S. Pat. No. 5,270,086, and Raeder-Devens, U.S. Pat. No. 6,726,712. Stents and stent delivery are also exemplified by the Sentinol® system, available from Boston Scientific Scimed, Maple Grove, Minn.

In some embodiments, stents can also be a part of a covered stent or a stent-graft. In other embodiments, a stent can include and/or be attached to a biocompatible, non-porous or semi-porous polymer matrix made of polytetrafluoroethylene (PTFE), expanded PTFE, polyethylene, urethane, or polypropylene.

In some embodiments, stents can also include a releasable therapeutic agent, drug, or a pharmaceutically active compound, such as described in U.S. Pat. No. 5,674,242, U.S. Ser. No. 09/895,415, filed Jul. 2, 2001, and U.S. Ser. No. 10/232,265, filed Aug. 30, 2002. The therapeutic agents, drugs, or pharmaceutically active compounds can include, for example, anti-thrombogenic agents, antioxidants, anti-inflammatory agents, anesthetic agents, anti-coagulants, and antibiotics.

In some embodiments, stents can be formed by fabricating a wire including a fractal structure, and knitting and/or weaving the wire into a tubular member. In some embodiments, medical implants other than stents include a fractal structure. Such medical implants can include cochlear implants, septal defect device plugs, AAA graph attachment stents, bone screws, Neuro aneurysm coils, venous valve support structures, heart valve support structure, placing leads, spinal implant support cages, hip replacement joints, inter uterine implants (e.g., for birth control). These medical implants can be formed of a bioerodable metal. The bioerodable metal can be magnesium, iron, zinc, or an alloy thereof. In some embodiments, the bioerodable metal can be a metallic iron or alloy thereof. For example, a medical implant could include an iron portion having an outermost surface having a fractal structure.

Furthermore, polymeric stents can also include a fractal structure to accelerate the degradation.

All publications, references, applications, and patents referred to herein are incorporated by reference in their entirety.

Other embodiments are within the claims.

What is claimed is:

1. An endoprosthesis comprising a member having a surface that includes a fractal structure.

2. The endoprosthesis of claim 1, wherein the member comprises a bioerodable material.

3. The endoprosthesis of claim 1, wherein the member comprises a bioerodable metal.

4. The endoprosthesis of claim 3, wherein the bioerodable metal comprises magnesium, zinc, iron, or an alloy thereof.

5. The endoprosthesis of claim 3, wherein the member comprises iron or an alloy thereof.

6. The endoprosthesis of claim 1, wherein the member comprises a bioerodable polymer.

7. The endoprosthesis of claim 6, wherein the bioerodable polymer is selected from the group consisting of polydioxanone, polycaprolactone, polygluconate, polylactic acid-polyethylene oxide copolymers, modified cellulose, collagen, poly(hydroxybutyrate), polyanhydride, polyphosphoester, poly(amino acids), poly-L-lactide, poly-D-lactide, polyglycolide, poly(alpha-hydroxy acid), and combinations thereof.

8. The endoprosthesis of claim 1, wherein the member comprises a non-biodegradable metal selected from the group consisting of stainless steels, platinum enhanced stainless steels, cobalt-chromium alloys, nickel titanium alloys, and combinations thereof.

9. The endoprosthesis of claim 1, wherein the surface is an outermost surface of the endoprosthesis.

10. The endoprosthesis of claim 1, further comprising a second material overlying the surface.

11. The endoprosthesis of claim 10, wherein the second material is selected from the group consisting of a tie layer, a biocompatible coating, a drug-eluting layer, a radiopaque metal or alloy, and combinations thereof.

12. The endoprosthesis of claim 11, wherein the second material is a drug-eluting layer.

13. The endoprosthesis of claim 1, wherein the fractal structure comprises a surface area that is greater than 5 times the surface area of a smooth surface having the same dimensions.

14. The endoprosthesis of claim 1, wherein the fractal structure comprises a cauliflower-like structure.

15. The endoprosthesis of claim 1, wherein the fractal structure comprises nanopits.

16. The endoprosthesis of claim 1, wherein the endoprosthesis is a stent.

17. An implant comprising:
a bioerodable material having a surface that includes a fractal structure.

18. The implant of claim 17, wherein the bioerodable material comprises iron or an alloy thereof.

19. The implant of claim 17, wherein the fractal structure comprises a surface area that is greater than 5 times the surface area of a smooth surface having the same dimensions.

20. The implant of claim 17, wherein the fractal structure comprises a cauliflower-like structure.

21. The implant of claim 17, wherein the fractal structure comprises nanopits.

22. The implant of claim 17, wherein the implant is selected from the group consisting of stents, cochlear implants, bone screws, neuron aneurism coils, septal defect plugs, venous valve support structures, pacing leads, spinal implant support structures, hip replacement joints, and inter uterine implants.

23. The implant of claim 17, wherein the implant is a stent.

24. A bioerodable stent comprising:
a bioerodable member that includes a surface having a fractal structure, the surface comprising iron or an alloy thereof.

25. The bioerodable stent of claim 24, wherein the fractal structure comprises a surface area that is greater than 5 times the surface area of a smooth surface having the same dimensions.

26. The bioerodable stent of claim 24, wherein the fractal structure comprises cauliflower-like structure.

27. The bioerodable stent of claim 24, wherein the fractal structure comprises nanopits.

28. The bioerodable stent of claim 24, wherein the surface is an outermost surface of the stent.
